(19) United States
(12) Patent Application Publication
(10) Pub. No.: US 2025/0120840 A1
(43) Pub. Date: Apr. 17, 2025

(54) CARPAL TUNNEL SPLINT

(71) Applicant: Safe Splints, LLC, Winter Park, FL (US)

(72) Inventor: Jeffrey A. Deren, Winter Park, FL (US)

(73) Assignee: Safe Splints, LLC, Winter Park, FL (US)

(21) Appl. No.: 18/912,886

(22) Filed: Oct. 11, 2024

Related U.S. Application Data
(60) Provisional application No. 63/543,632, filed on Oct. 11, 2023.

(51) Int. Cl. A61F 5/058

(52) U.S. Cl. CPC A61F 5/05866; A61F 5/05816

(57) ABSTRACT

Carpal tunnel splint. A splint device referenced herein includes a rigid board inflatable by way of a first inflation valve, and a soft inflatable bladder positioned relative to the rigid board and inflatable by way of a second inflation valve, wherein the rigid board is configured to be positioned along a volar portion of a hand, a wrist, and an arm of a user, and wherein the soft inflatable bladder is configured to be positioned at a palm of the hand.

### PRIORITY

The present application is related to, and claims the priority benefit of, U.S. provisional patent application Ser. No. 63/543,632, filed Oct. 11, 2023, the contents of which are incorporated herein directly and by reference in their entirety.

### BACKGROUND

Carpal tunnel syndrome is a very common condition, occurring when the median nerve becomes pressed or squeezed at the wrist. This causes pain, numbness, weakness, and potential swelling.

Currently, all night time carpal tunnel splints are big and bulky. Furthermore, the wrist tends to be held in 20-30 degrees of extension, which is the “functional position” (such as when using a computer mouse or typing), but might not be ideal for comfort in a patient with carpal tunnel symptoms. As such, a splint that provides comfort and relief while sleeping would be well received in the marketplace.

### BRIEF SUMMARY

The present disclosure includes disclosure of a splint device, comprising a rigid board inflatable by way of a first inflation valve, and a soft inflatable bladder positioned relative to the rigid board and inflatable by way of a second inflation valve, wherein the rigid board is configured to be positioned along a volar portion of a hand, a wrist, and an arm of a user, and wherein the soft inflatable bladder is configured to be positioned at a palm of the hand.

The present disclosure includes disclosure of a splint device, further comprising a dorsal pad configured to be positioned along a dorsal portion of the hand, the wrist, and the arm.

The present disclosure includes disclosure of a splint device, further comprising flexible bands extending between the dorsal pad and the rigid board.

The present disclosure includes disclosure of a splint device, wherein when the device is worn by a user and the rigid board and the soft inflatable bladder are not inflated, the wrist would not be positioned at an angle relative to the arm.

The present disclosure includes disclosure of a splint device, wherein when the device is worn by a user and the rigid board and the soft inflatable bladder are inflated, the hand would be positioned at an angle relative to the arm between 0° and about 20°.

The present disclosure includes disclosure of a splint device, further comprising one or more cross bands used to connect two flexible bands of the flexible bands to one another.

The present disclosure includes disclosure of a splint device, wherein the dorsal pad extends to the rigid board.

The present disclosure includes disclosure of a splint device, further comprising a glove cover configured to fit around the rigid board and the dorsal pad.

The present disclosure includes disclosure of a splint device, further comprising a glove cover extending around the rigid board and around the hand.

The present disclosure includes disclosure of a splint device, wherein the glove cover is configured to provide compressive pressure to the hand, the wrist, and the arm.

The present disclosure includes disclosure of a splint device, wherein when the device is worn about the hand, the wrist, and the arm of the user, the rigid board can be inflated to become rigid, and the soft inflatable bladder can be inflated to adjust an angle of the hand relative to the arm from 0° to about 20°.

The present disclosure includes disclosure of a splint device, configured to provide relief from carpal tunnel syndrome discomfort.

The present disclosure includes disclosure of a splint device, as shown and/or described herein.

The present disclosure includes disclosure of a splint device configured to provide relief from carpal tunnel syndrome discomfort, as shown and/or described herein.

The present disclosure includes disclosure of a method for providing relief from carpal tunnel syndrome discomfort, as shown and/or described herein.

The present disclosure includes disclosure of a method for providing relief from carpal tunnel syndrome discomfort, comprising placing a device of the present disclosure over the hand of a user, inflating the rigid board of the device to make said rigid board rigid, and inflating the soft inflatable bladder of the device to change an angle of the back of the hand relative to the arm between 0° and about 20°.

The present disclosure includes disclosure of a method for providing relief from carpal tunnel syndrome discomfort, comprising placing a device of the present disclosure over the hand of a user, and inflating the soft inflatable bladder of the device to change an angle of the back of the hand relative to the arm between 0° and about 20°.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1 shows portions of a splint device positioned relative to the hand, wrist, and arm, without inflation of the soft inflatable bladder, according to an exemplary embodiment of the present disclosure;

FIG. 2 shows portions of a splint device positioned relative to the hand, wrist, and arm, with inflation of the soft inflatable bladder, according to an exemplary embodiment of the present disclosure;

FIG. 3 shows a splint device positioned relative to the hand, wrist, and arm, with inflation of the soft inflatable bladder, according to an exemplary embodiment of the present disclosure;

FIG. 4 shows a splint device positioned relative to the hand, wrist, and arm, according to an exemplary embodiment of the present disclosure; and

FIG. 5 shows a splint device positioned relative to the hand, wrist, and arm, according to an exemplary embodiment of the present disclosure.

As such, an overview of the features, functions and/or configurations of the components depicted in the various figures will now be presented. It should be appreciated that not all of the features of the components of the figures are necessarily described and some of these non-discussed features (as well as discussed features) are inherent from the figures themselves. Other non-discussed features may be inherent in component geometry and/or configuration. Furthermore, wherever feasible and convenient, like reference numerals are used in the figures and the description to refer to the same or like parts or steps. The figures are in a simplified form and not to precise scale.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

The present disclosure includes disclosure of a carpal tunnel cloud splint configured to provide relief while sleeping, for example, from those suffering from carpal tunnel syndrome. Comfort and pain relief are key factors in connection with said splints.

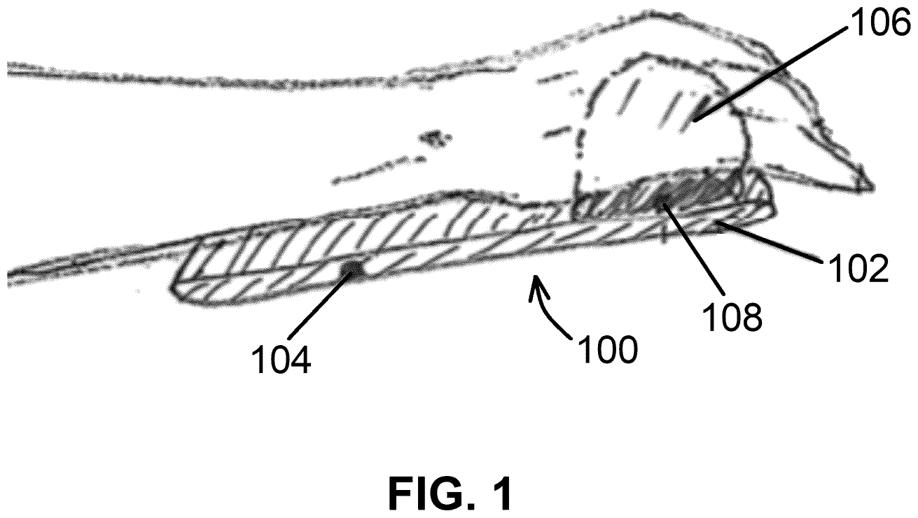

An exemplary carpal tunnel cloud splint device 100 of the present disclosure is shown in FIG. 1. As shown therein, device 100 comprises a rigid inflatable board 102 configured to be positioned along volar/palmar portions of the hand, wrist, and arm. Said board 102 can be inflated by way of an inflation valve 104 coupled thereto.

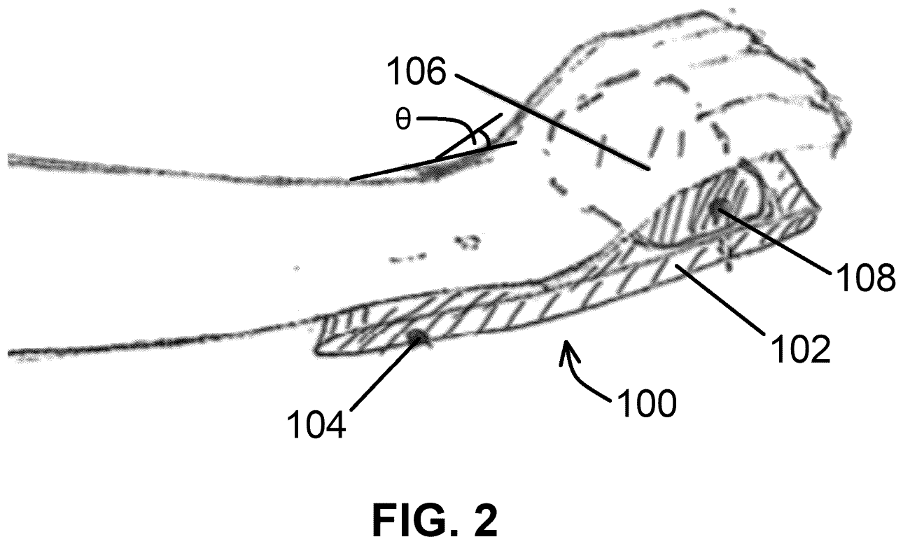

As shown in FIG. 1, for example, an exemplary device 100 of the present disclosure also comprises a soft inflatable bladder 106 positioned relative to board 102, whereby when the device 100 is worn by a user, the inflatable bladder 106 would be positioned adjacent the user's palm. FIG. 1 shows the inflatable bladder in an uninflated configuration, and FIG. 2 shows the inflatable bladder 106 in an inflated configuration. Inflation of inflatable bladder 106, when device is worn, would result in changing the wrist angle of extension relative to the arm, such as a degree of wrist extension up to 20° in at least one embodiment. Inflatable bladder 106 can be separately inflated by way of one or more inflation valves 108 coupled thereto. In at least one embodiment, board 102 and inflatable bladder 106 are coupled to one another.

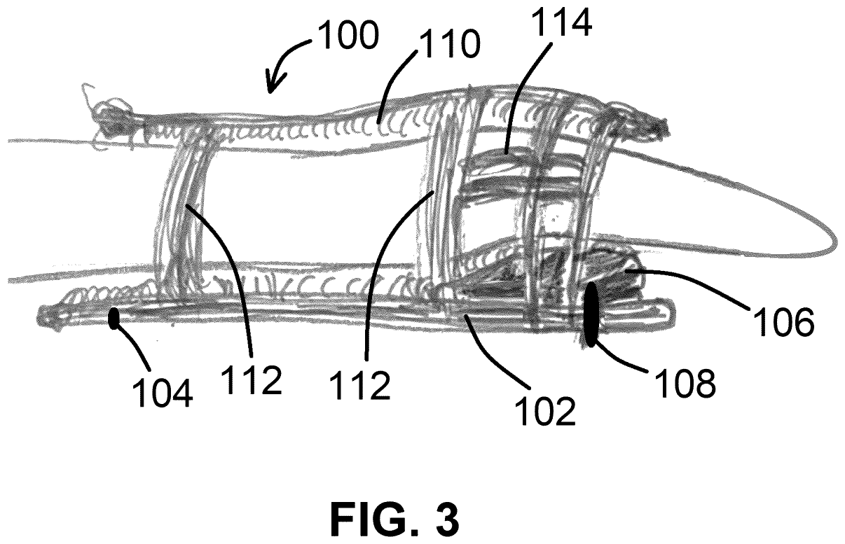

FIG. 3 shows a device 100 of the present disclosure with additional parts/components. As shown therein, devices 100 can further comprise a dorsal pad 110 configured to fit along at least a dorsal portion of the hand, wrist, and arm. In at least one embodiment, flexible bands 112 coupled to board 102 and dorsal pad 110 can be used so that some compressive pressure can be placed upon the hand, wrist, and arm, even while board 102 and inflatable bladder 106 are inflated. Cross bands 114 can also be used to connect flexible bands 112 to one another, such as shown in FIG. 3.

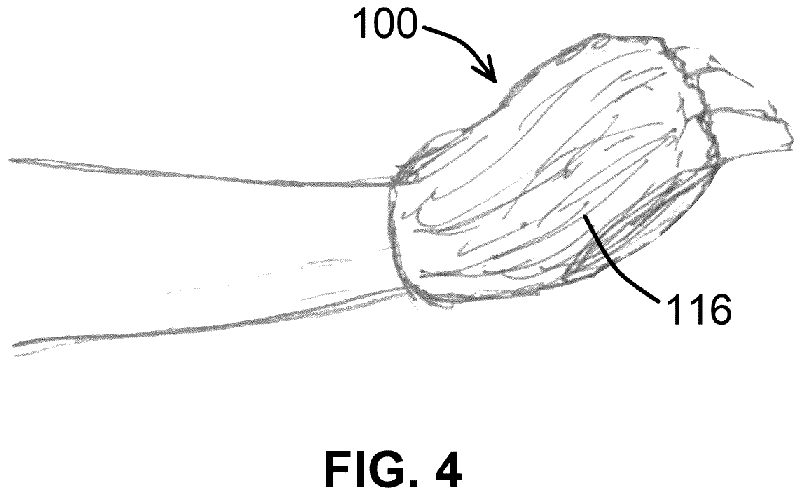

FIG. 4 shows another device of the present disclosure, configured as a soft fingerless glove. Within said glove (not shown in FIG. 4 but shown in other figures) would include board 102 and inflatable bladder 106, and dorsal pad 110 can be considered as extending to each side of the hand, wrist, and arm, contacting or encasing board 102. Alternatively, such an embodiment would include board 102 and inflatable bladder 106, covered by a fingered or fingerless glove cover 116, whereby fingered or fingerless glove would encase board 102 and inflatable bladder 106. Glove cover 116 can be configured as a compressive cuff, negating the need of flexible bands 112, or glove cover 116 cannot provide any meaningful compressive pressure, so that flexible bands 112 would be needed to connect board 102 to dorsal pad 110. In at least one embodiment, glove cover 116 comprises fur, lamb's wool, or another soft material to increase overall comfort when said glove cover 116 is worn by a user. Materials can also be sweat-wicking or naturally cooling. In some embodiments, device 100 can be unfolded and placed over the hand, such as shown in FIG. 4.

In use, and when worn, device 100 of the present disclosure would cover at least part of a user's palm, wrist, and part of the user's arm, such as shown in FIGS. 3 and 4. When device 100 is positioned, board 102 and inflatable bladder 106 can each be inflated to provide rigidity to said board 102 and to change the angle of the dorsal side of the hand relative to the dorsal side of the arm. By doing so, and by providing an angle of up to about 20° (as identified by the angle symbol in FIG. 2, for example), said user can experience relief and comfort from carpal tunnel syndrome discomfort.

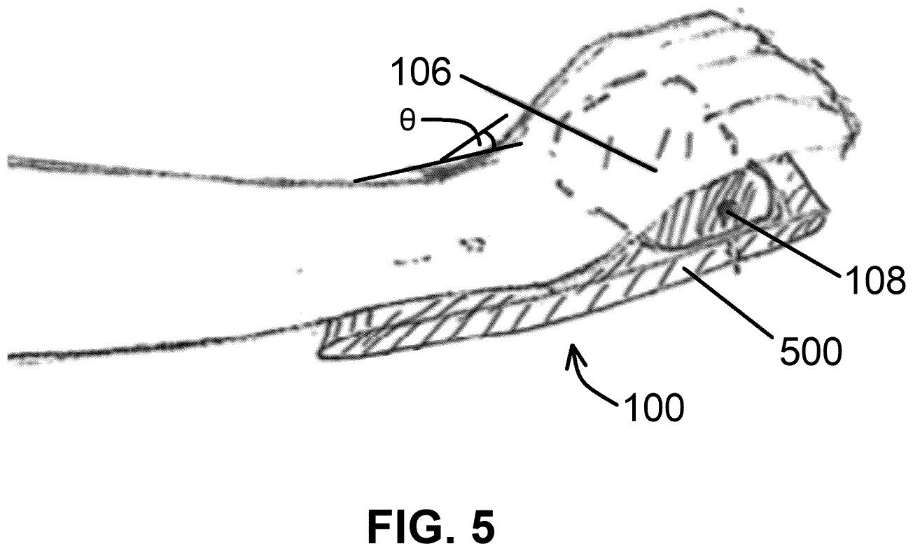

An exemplary carpal tunnel cloud splint device 100 of the present disclosure is shown in FIG. 5. As shown therein, device 100 comprises a rigid plate 500 that is not inflatable, instead of a rigid inflatable board 102, whereby rigid plate 500 is configured to be positioned along volar/palmar portions of the hand, wrist, and arm.

As shown in FIG. 5, for example, an exemplary device 100 of the present disclosure also comprises a soft inflatable bladder 106 positioned relative to plate 500, whereby when the device 100 is worn by a user, the inflatable bladder 106 would be positioned adjacent the user's palm. FIG. 5 shows the inflatable bladder 106 in an inflated configuration. Inflation of inflatable bladder 106, when device is worn, would result in changing the wrist angle of extension relative to the arm, such as a degree of wrist extension up to 20° in at least one embodiment. Inflatable bladder 106 can be separately inflated by way of an inflation valve 108 coupled thereto.

A unique feature of devices 100 of the present disclosure is the ability of a user to adjust the degree of wrist extension between zero and about 20° to give the best level of comfort while sleeping or otherwise when relief from discomfort is desired.

It is particularly noted that devices 100 of the present disclosure are suitable for nighttime use, providing relief from carpal tunnel syndrome symptoms to improve overall sleep comfort.

While various embodiments of devices for and methods for using the same have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. A splint device, comprising:
a rigid board inflatable by way of a first inflation valve; and
a soft inflatable bladder positioned relative to the rigid board and inflatable by way of a second inflation valve;
wherein the rigid board is configured to be positioned along a volar portion of a hand, a wrist, and an arm of a user; and
wherein the soft inflatable bladder is configured to be positioned at a palm of the hand.

2. The splint device of claim 1, further comprising:
a dorsal pad configured to be positioned along a dorsal portion of the hand, the wrist, and the arm.

3. The splint device of claim 2, further comprising:
flexible bands extending between the dorsal pad and the rigid board.

4. The splint device of claim 3, wherein when the device is worn by a user and the rigid board and the soft inflatable bladder are not inflated, the wrist would not be positioned at an angle relative to the arm.

5. The splint device of claim 3, wherein when the device is worn by a user and the rigid board and the soft inflatable bladder are inflated, the hand would be positioned at an angle relative to the arm between 0° and about 20°.

6. The splint device of claim 3, further comprising:
one or more cross bands used to connect two flexible bands of the flexible bands to one another.

7. The splint device of claim 2, wherein the dorsal pad extends to the rigid board.

8. The splint device of claim 2, further comprising:
a glove cover configured to fit around the rigid board and the dorsal pad.

9. The splint device of claim 1, further comprising a glove cover extending around the rigid board and around the hand.

10. The splint device of claim 9, wherein the glove cover is configured to provide compressive pressure to the hand, the wrist, and the arm.

11. The splint device of claim 1, wherein when the device is worn about the hand, the wrist, and the arm of the user, the rigid board can be inflated to become rigid, and the soft inflatable bladder can be inflated to adjust an angle of the hand relative to the arm from 0° to about 20°.

12. The splint device of claim 1, configured to provide relief from carpal tunnel syndrome discomfort.

13. A method for providing relief from carpal tunnel syndrome discomfort, comprising:
placing the device of claim 1 over the hand of a user;
inflating the rigid board of the device to make said rigid board rigid; and
inflating the soft inflatable bladder of the device to change an angle of the back of the hand relative to the arm between 0° and about 20°.

14. A method for providing relief from carpal tunnel syndrome discomfort, comprising:
placing the device of claim 1 over the hand of a user; and
inflating the soft inflatable bladder of the device to change an angle of the back of the hand relative to the arm between 0° and about 20°.
